(19) United States
(12) Patent Application Publication
(10) Pub. No.: US 2013/0152978 A1
(43) Pub. Date: Jun. 20, 2013

(54) CONTACT LENS CLEANING APPARATUS AND METHOD

(71) Applicants: Jacob Allred, Houston, TX (US); Majid Abbasi, Everett, MA (US); Jacob Morisse, Provo, UT (US)

(72) Inventors: Jacob Allred, Houston, TX (US); Majid Abbasi, Everett, MA (US); Jacob Morisse, Provo, UT (US)

(21) Appl. No.: 13/693,548

(22) Filed: Dec. 4, 2012

Related U.S. Application Data
(60) Provisional application No. 61/576,271, filed on Dec. 15, 2011.

(51) Int. Cl. A61L 12/02

(52) U.S. Cl. CPC A61L 12/026
USPC 134/34; 134/184

(57) ABSTRACT

A combination of ultrasonic waves causing microcavitation bubbles to form between the surface of a contact lens and an attached contaminant layer formed by microbes thereon initiates separation. However, a vigorous convective flow takes advantage of any localized breaking and separating of the contaminant layer and immediately imposes a hydrodynamic drag force on the pieces of the contaminant layer that may be separated, thus leading to greater separation, pealing, and tearing of the contaminant layer away from the surface. Thus, the combination has proven much more effective than either feature alone.

The present invention may be embodied in other specific forms without departing from its fundamental functions or essential characteristics. The described embodiments are to be considered in all respects only as illustrative, and not restrictive. All changes which come within the meaning and range of equivalency of the illustrative embodiments are to be embraced within their scope.

### RELATED APPLICATIONS

This application: claims the benefit of co-pending U.S. Provisional Patent Application Ser. No. 61/576,271, filed on Dec. 15, 2012; which is hereby incorporated by reference.

### BACKGROUND

1. The Field of the Invention

This invention pertains to cleaning systems and, more particularly, to novel systems and methods for cleaning contact lenses.

2. The Background Art

Spectacles or eyeglasses have given way in many instances to contact lenses. The original hard contact lenses of decades past have largely given way to soft contact lenses. These soft contact lenses provide not only softer, more flexible plastics but also plastics that are semi-permeable. These semi-permeable or gas-permeable lenses permit oxygen to pass through the matrix of the lens material and thus oxygenate the surface of the eye on which the contact rests during use.

Nevertheless, soft contact lenses or gas-permeable contact lenses are not without their own problems limitations. For example, users are typically advised on a cleaning and wearing protocol for the lenses. Severe injury or blindness can occur if the protocol is not followed. Sometimes, lenses may be torn by too vigorous handling.

Regardless, lenses tend to build up a film of contaminants. Contaminants are commonly deposited on surfaces of the lens and eventually develop growth into the interstices or openings within the plastic matrix of the lens itself. These contaminants are supposedly removed by cleaning.

However, it has been found that even the best mechanical and chemical cleaning techniques can typically remove particulate matter that presents unevenness and the like. However, these are often insufficient to completely clean up the well-developed, smoother layer of contaminants. Current washers, liquids, chemicals, and the like render the lenses caustic and unwearable. That is, if harsh chemicals are used, then it is imperative yet difficult to return the pH level of the lens to a suitable level in order to make them comfortable and not damaging to the eyes of the user.

It would be an improvement in the art to discover and implement a method for more effective cleaning of contact lenses that is rapid, safe, and simple.

### SUMMARY OF THE INVENTION

In view of the foregoing, in accordance with the invention as embodied and broadly described herein, an apparatus and method implementing both ultrasonic cavitation and microcavitation between the lens and the contaminant layer. In addition, a lateral shear flow by a convective current assists in the mechanical removal of the contaminant layer.

Whereas abrasion has been shown to be damaging and ineffective, an initial imposition of ultrasonic waves results in microcavitation in the contaminant layer, and particularly between the contact lens and the contaminant layer where differences in material properties tend to be emphasized by the ultrasonic waves. Moreover, by imposing a lateral shear by convective currents passing over the surface of the contact lens, any fracturing or rupture of the contaminant layer is immediately amplified by the high shear in the boundary layer of the passing, convective fluid flow. In this way, the contaminant layer is separated from the surface of the contact lens and mechanically removed.

In one implementation, a method for cleaning contact lenses may provide a basket for containment, a lens having a contaminant layer developing on it, and a containment vessel or well. Placing the lens in the basket and the basket in a container containing a liquid is followed by creating ultrasonic waves in the liquid by operation of an ultrasonic transducer moving a wall of the container, typically its floor.

This creates micro cavitation tending to cause bubbles between the contaminant layer and the lens. Passing a convective flow across a surface of the lens causes fluid drag, lifting the contaminant layer away, peeling it back, and tearing it off the lens to which it has attached. The flow carries the torn pieces of the contaminant film from the lens. The convective flow has been found best if operating in a turbulent regime. However, more important is stripping away and thinning the boundary layer sufficiently to expose the contaminant layer to the fluid drag, which gently but firmly lifts, peels, and tears it away. This is more difficult with laminar flow.

The ultrasonic waves are selected to be effective to create micro-cavitation lifting the contaminant layer away from the lens. Normally, this might still allow a re-laying of the layer, and re-attachment. However, the convective flow is effective to create dynamic head pressure under the layer at points of disruption and hydrodynamic drag against any portion of the contaminant layer separated by the ultrasonic waves from the lens. The hydrodynamic drag is sufficient to engage the tensile strength of the contaminant layer, causing separated portions to peel neighboring regions away from the surface of the lens.

Typically, the contaminant layer is bonded to the lens by mechanical extension of the contaminant layer into the matrix of a polymer forming the lens. The ultrasonic waves are introduced at a frequency and power effective to create bubbles forcing the contaminant layer from the lens at localized places. The bubbles create tensile stresses in the contaminant layer, lifting, and sometimes rendering or rupturing the layer. Separating the first portion of the contaminant layer from the lens by the hydrodynamic drag creates tensile stresses in the contaminant layer to support continued peeling and eventual tearing.

An apparatus for cleaning contact lenses may include a well containing a liquid, with a basket submerged in the liquid and holding an article such as a lens, having a surface to be cleaned of protein, biofilms, or other contaminants. A contaminant layer is typically disposed as a contiguous film on the surface and bonded thereto by a mechanical engagement, as well as other adhesive forces.

A transducer creates ultrasonic waves in the liquid impinging on the surface. A convector creates convection currents passing along the surface. The transducer provides the power and motion to break loose the bonding of the contaminant layer by cavitating (vaporizing) the liquid proximate the surface in response to a pressure wave due to movement at a comparatively high speed. The convector, first lifts, then begins to peel, and eventually tears the contaminant layer from the surface by applying fluid drag to any portion of the contaminant layer extending out of the boundary layer (typically a comparatively thin boundary layer in accordance with high Reynolds' number flows in the turbulent regime) into the convection current.

The frequency of the waves is ultrasonic, and has been found to serve best in a range of from about 30 to about 120 kilohertz. A Piezoelectric transducer was used in experiments to create the waves. The convector was a motor driving an impeller (propeller, fan, etc.) The liquid was water, treated with a combination of a cleaning agent such as a surfactant, a bactericide, and a salt in the best or most effective configurations.

It appears that a contact lens is formed of a polymer having strands defining interstices therebetween; and the contaminant layer extends into the interstices. Thus, in addition to any adhesive properties of the contaminant biofilm, it appears to mechanically grow into those interstices, maintaining a mechanical adherence as well. Other cleaning methods and apparatus have not been successful at stripping away such contaminant films.

### BRIEF DESCRIPTION OF THE DRAWINGS

The foregoing and other objects and features of the present invention will become more fully apparent from the following description and appended claims, taken in conjunction with the accompanying drawings. Understanding that these drawings depict only typical embodiments of the invention and are, therefore, not to be considered limiting of its scope, the invention will be described with additional specificity and detail through use of the accompanying drawings in which:

FIG. 1 is a frontal cross sectional view of a cleaning apparatus in accordance with the invention;

FIG. 2 is a frontal cross sectional view of the core of the apparatus of FIG. 1 removed from the outer housing in order to illustrate certain operational components therein;

FIG. 3 is an upper perspective view of the apparatus of FIG. 1, having the core of FIG. 2 removed and illustrating the central well that receives the core;

FIG. 4 is a perspective view of the apparatus of FIG. 1, having the core and its lid to which it is placed in the housing;

FIG. 5 is a side elevation view from the right side of the apparatus of FIG. 1;

FIG. 6 is a cross sectional view of the arrangement of a contact lens attached to a cornea of an eye and illustrating the location of a contaminant layer attached to the contact lens;

FIG. 7 is a schematic diagram illustrating the operable components and effects of an apparatus and method in accordance with the invention for cleaning contact lenses;

FIG. 8 is a schematic diagram of the activities at the interface between the contaminant layer and the contact lens during the cleaning process;

FIG. 9 is a magnified view of a contaminant layer of a contact lens during the cleaning process in accordance with the invention;

FIG. 10 is an image illustrating the polymer chains and the interstices therebetween in a layer of a polymer as viewed in a scanning electron micrograph of such a layer, thereby illustrating the interstitial spaces in which microbiological organisms may bind themselves;

FIG. 11 is a sequence of activity during one embodiment of a cleaning process;

FIG. 11A is a schematic diagram illustrating the condition of a contact lens surface covered by a contaminant layer;

FIG. 11B is a schematic diagram illustrating microcavitation tending to cause localized separation of the contaminant layer of the contact lens;

FIG. 11C is a schematic diagram illustrating rupture of the contaminant layer; and

FIG. 11D is a schematic diagram illustrating the shearing force of the boundary layer of a convective flow tearing away the contaminant layer from the surface of a contact lens.

### DETAILED DESCRIPTION OF THE PREFERRED EMBODIMENTS

It will be readily understood that the components of the present invention, as generally described and illustrated in the drawings herein, could be arranged and designed in a wide variety of different configurations. Thus, the following more detailed description of the embodiments of the system and method of the present invention, as represented in the drawings, is not intended to limit the scope of the invention, but is merely representative of various embodiments of the invention. The illustrated embodiments of the invention will be best understood by reference to the drawings, wherein like parts are designated by like numerals throughout.

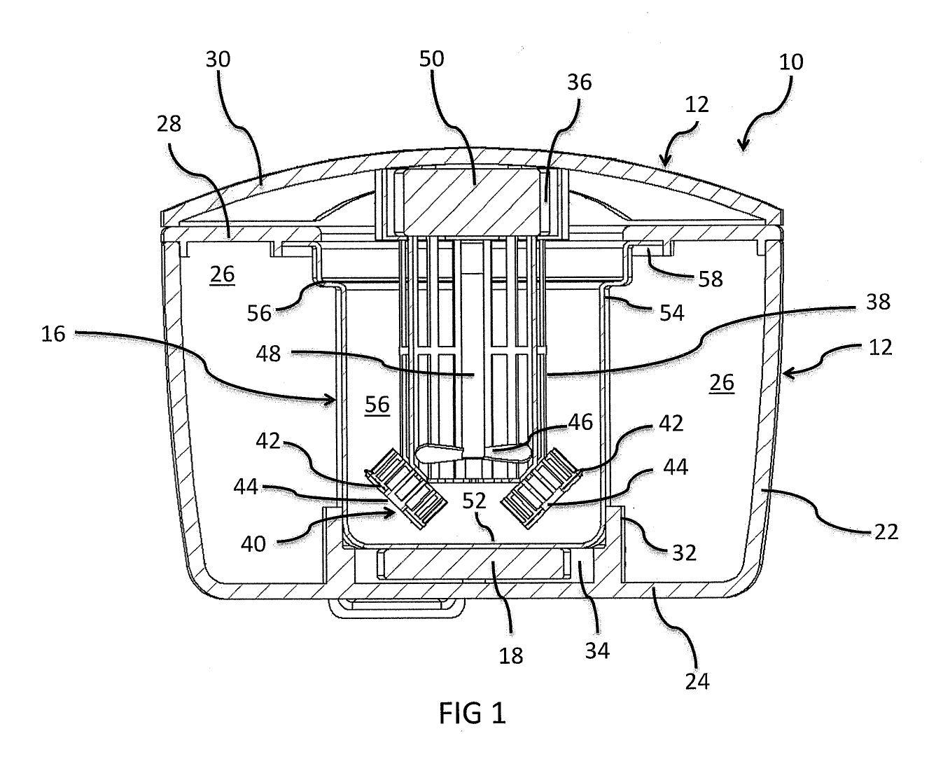

Referring to FIG. 1, an apparatus 10 or system 10 in accordance with the invention may include a housing 12. Inside the housing 12 is contained a convection drive assembly 14 or assembly 14 responsible to provide convective shear forces across a surface. In the illustrated embodiment, the assembly 14 or drive 14 is contained within a well 16 within the housing 12.

At the bottom of the well 16 is placed a transducer 18. Typically, the transducer 18 may be a piezoelectric transducer changing dimension with each change in voltage.

In certain embodiments, the well 16 may be the entire housing 12. Nevertheless, in other embodiments it may be useful to leave space between the outer wall of the well 16 and the housing 12. For example, in order to insulate against sound transmission, thermal variations, or the like, including aesthetic reasons or benefits, the well 16 may or may not be coincident with the outer wall of the housing 12.

In the illustrated embodiment, a transducer 18 is responsible for generating ultrasonic waves within the liquid inside the well 16. The ultrasonic waves result in localized cavitation and microcavitation on the surface of the contact lens within the well 16.

The transducer 18 and other electronic and electrical components of system 10 in accordance with the invention may be housed within a chamber 20, isolating electrical and electronic components from the liquids, such as water, that may be spilled to the electronics or to the transducer 18 from the well 16.

The outer wall 22 of the housing 12 may be spaced from the well 16 in order to provide isolation as described hereinabove. Nevertheless, in certain embodiments, aesthetically pleasing shapes may be designed into the housing 12, thus requiring some difference between the location and size of the wall 22 and that of the well 16.

In the illustrated embodiment, the floor 24 of the housing 12 may support various other structures, including the well 16. For example, the housing 12 may include a cavity 26 containing a surrounding ambient air, another liquid, insulation against thermal transmission, insulation against sound transmission, or the like.

In certain embodiments, a cover 28 may form a structural alignment device 28 in order to position and maintain the well 16 within the housing 12. The cover 28 operates as a mechanical restraint holding the well 16 in place. It may have a clearance, an elastomeric gasket for vibration isolation or the like with respect to the well 16.

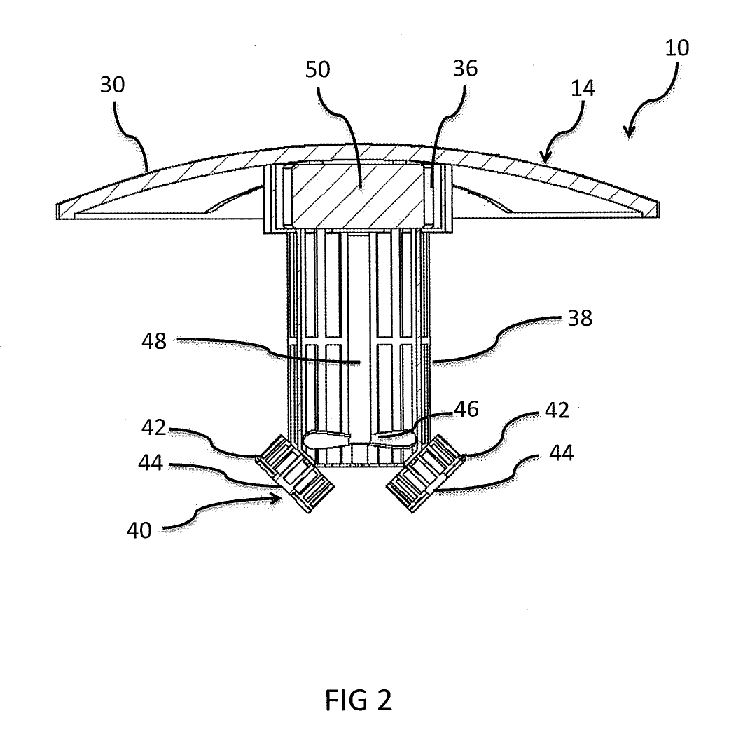

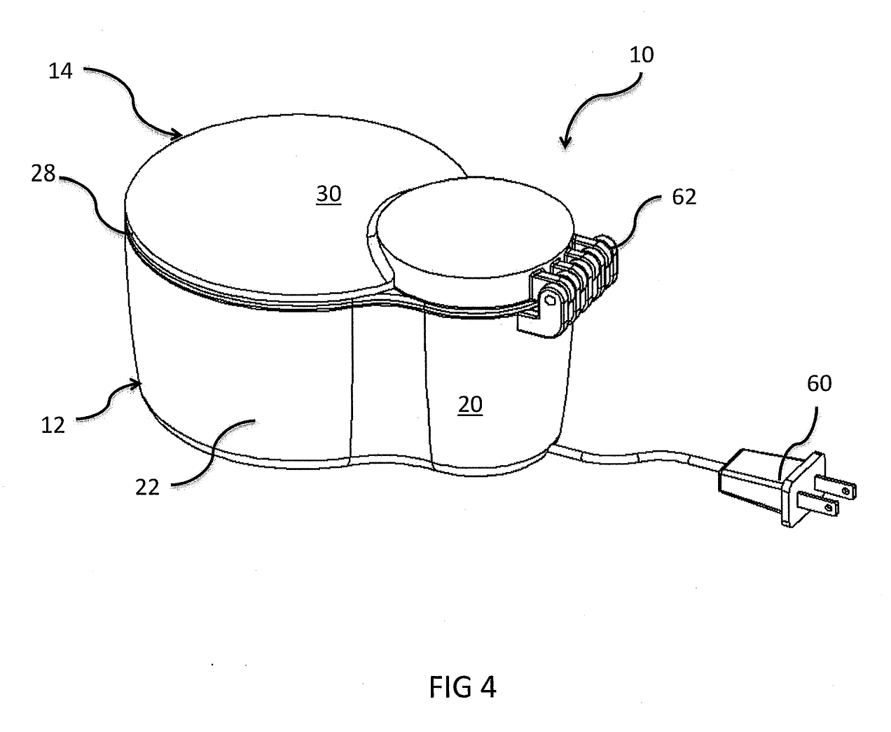

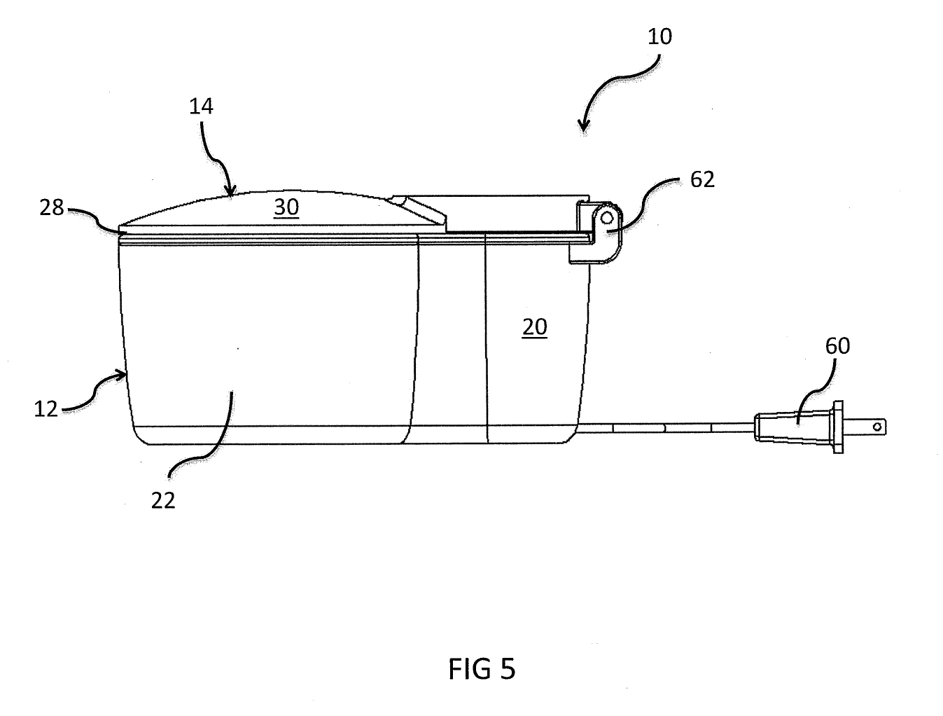

Referring to FIGS. 1-5 in general, while continuing to refer to FIG. 1 specifically, the lid 30 may form a backbone 30 for the convection drive assembly 14. Similarly, the lid 30 operates to form the closure 30 of the housing 12 over the well 16. In the illustrated embodiment, the lid 30 may be hinged or otherwise removable from the remainder of the housing 12 in order to remove the convection drive assembly 14 for use, for loading, or the like.

Opposite the lid 30, a mount 32 for the well 16 may be formed contiguously with the floor 24 of the housing 12. The mount 32 may operate to register the well 16 and to constrain the well 16 opposite the cover 28. Thus, the well 16 may be removable. In other embodiments, the well 16 may actually be molded integrally with the housing 12. However, in the illustrated embodiment, it has been found effective to render the well 16 removable. Thus, in operation, the well 16 may be captured or restrained between the mount 32 therebelow and the cover 28 above. Alternatively, the well 16 may be isolated from the mount 32 to enhance vibration. In yet another embodiment the transducer may be mounted to the well 16 and provide its exclusive support with respect to the floor 24.

A cavity 34 in the housing 12 and within the envelope of the mount 32 may contain the transducer 18. It is important to keep the transducer 18 in intimate mechanical contact with the well 16. Accordingly, in certain embodiments, the transducer 18 may actually be bonded to the well 16, in order to transmit mechanical vibrations directly to the well 16, and any contained fluid therein. Thus, some cavity 34 or relief 34 will typically be required in order to provide the transducer 18 the required space to operate. Moreover, the cavity 34 may typically separate the transducer 18 from an outer portion of the housing 12, such as keeping the transducer away from the floor 24 in order to not provide generalized vibration of the housing 12.

In certain embodiments the mount 32 below the well 16 may be absent entirely. In other embodiments, it may include soft, elastic, or even foamed, polymeric isolators in order to minimize vibration of the housing 12 through the floor 24. Minimizing or eliminating the vibrational response of the mount 32 below the well 16 in response to the ultrasonic vibrations of the transducer 18 may be engineered to minimize vibration of the housing 12.

The assembly 14 may include a mount 36 that will hold a motor 50 or other motive system 50. Typically, a cage 38 may extend from the mount 36 or elsewhere from the lid 30 that forms the backbone 30 for the convection drive assembly 14. The cage 38 may operate both as a protection from moving parts, and for those moving parts, as well as a mount for baskets 40 to contain contact lenses 72. In the illustrated embodiment, a basket 40 may be provided with a lid 42, typically secured by a hinge 44 to the basket 40. Thus, the basket 40 may be opened by pivoting the lid 42 about the hinge 44 in order to open the basket 40 for additional removal of the materials to be cleaned.

Typically the impeller 46 may be of any suitable type. In the illustration, the impeller 46 may operate at the end of a shaft 48, all protected within the cage 38. In this way, the cage 38 protects each of the components therein from being touched by a user. Likewise, the shaft 48 with the impeller 46 on it may be rotated by the motor 50 without interference.

An impeller 46 may be ducted, enclosed, or the like, rendering it a pump 46, other mechanisms for inducing a vigorous flow may include magnetic stirrers, a buoyant plume, a bubbler, or other motive source. In certain embodiments, the motor 50 may be of any suitable type. However, it has been found suitable to use a direct current (DC) motor 50.

The motor 50 may be connected in a fixed relationship to the shaft 48. Typically, the shaft 48 may be connected by an assembly, such as a sleeve or the like, to be driven by the motor 50. Likewise, at an end of the shaft 48 opposite to the motor 50, the impeller 46 may be configured as a propeller, fan, or the like suitable for inducing invigorous flows in forced convection to pass through the baskets 40. Thus, the baskets 40 may be formed as a grid of members spaced apart, such as rods, or structural elements promoting the passage therethrough of substantial flows of liquid driven by the impeller 46 on the shaft 48.

The floor 52 of the well 16 is fitted to the mount 32, or the well 16 may attach directly to the transducer 18. As described hereinabove, the floor 52 may be isolated from the mount 32 or from the transducer 18 by an elastomeric element isolating the motion of the floor 52 of the well 16 away from the mount 32, the housing 12 from the transducer, or both. That is, for example, the transducer 18 will vibrate ultrasonically, driving the floor 52 of the well 16 with it. In fact, that is the function of the transducer 18, to vibrate the floor 52 at an ultrasonic frequency in order to generate waves in the liquid contained in the well 16.

Thus, it may be advisable to minimize sound, motion, and the like to isolate the well 16 from the mount 32. In fact, the transducer 18 itself may be the exclusive bottom mount 32 for the well 16. Nevertheless, in certain embodiments it has been found suitable to mold the housing 12, including the mount 32 from a monolithic, homogeneous plastic. Thus, it has not always been found necessary to isolate the well 16 vibrationally from the mount 32, but the best vibration is available when the transducer 18 is the exclusive lower mount 32 for the well 16.

The wall 54 of the well 16 may be sized and shaped in order to surround the cage 48 and the remaining portions of the convection drive assembly 14 that must be immersed within the well 16. Similarly, the wall 54 may enclose a cavity 56 or interior 56 suitable for receiving and enclosing the baskets 40, the cage 38, and so forth. Typically, the cavity 56 or interior 56 of the well 16 is filled with a liquid, such as water.

The water may include chemicals in order to provide a physiological saline-like pH suitable for the cleaning process. Likewise, other cleaning chemicals known in the art may be used as a matter of course. Thus, the vibration of the transducer 18, transmitted by and through the floor 52 of the well 16, generates ultrasonic waves passing through the liquid held in the cavity 56 or interior 56 of the well 16.

A shoulder 58 may be formed in the wall 54 of the well 16 to add increased section modulus to the well 16. For example, the wall 54, having a change in section, a change in direction, or both may present one or more shoulders 58 adding a stiffening rim to the well 16. Likewise, the shoulder 58 may fit under the lid 28, thus capturing the well 16 between the mount 32 and the lid 28.

A power source 60, such as a power line 60, may carry electrical current to drive the motor 50. In certain embodiments, the power source 60 may be a line transmitting wall current at line voltage to a power supply within the cavity 26 in the housing 12. For example, wall current is ubiquitous throughout the civilized world. Although the cyclic frequency in voltage will vary between countries, wall current and voltage are well established.

In some embodiments, it may be advisable to reduce the voltage within the system 10 and particularly the voltage at which the motor 50 and transducer 18 operate. Therefore, it may be advisable to provide a power conditioning system within the cavity 26 of the housing 12 in order to provide to the transducer 18 and motor 50 the appropriate voltage and current. In certain embodiments, each of the transducer 18 and motor 50 may operate at line current. However, typically, this will not be the case. Instead, the transducer 18 and motor 50 will operate at much lower voltage than line voltage.

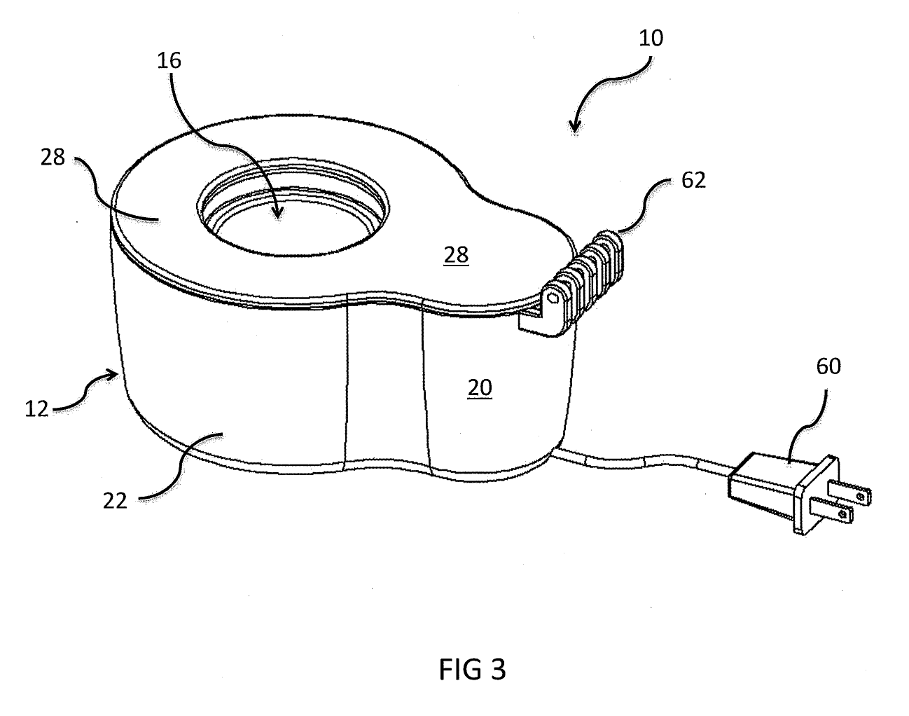

Referring to FIGS. 3-5, while continuing to refer to FIGS. 1-5, a system 10 in accordance with the invention may include a hinge 62 on the lid 30 securing the lid 30 to the remainder of the housing 12. That is, the housing 12 includes portions surrounded by the floor 24 and wall 22 as well as the lid 30. Thus, the lid 30 may be secured by gravity, a snaplock or detent, no hinge, or a hinge 62 to the wall 22. The lid 30 may be lifted, or pivoted to an open position, exposing the internal portions of the convection drive assembly 14, such as the motor 50, the cage 38, the baskets 40, the shaft 48, the impeller 46, and so forth.

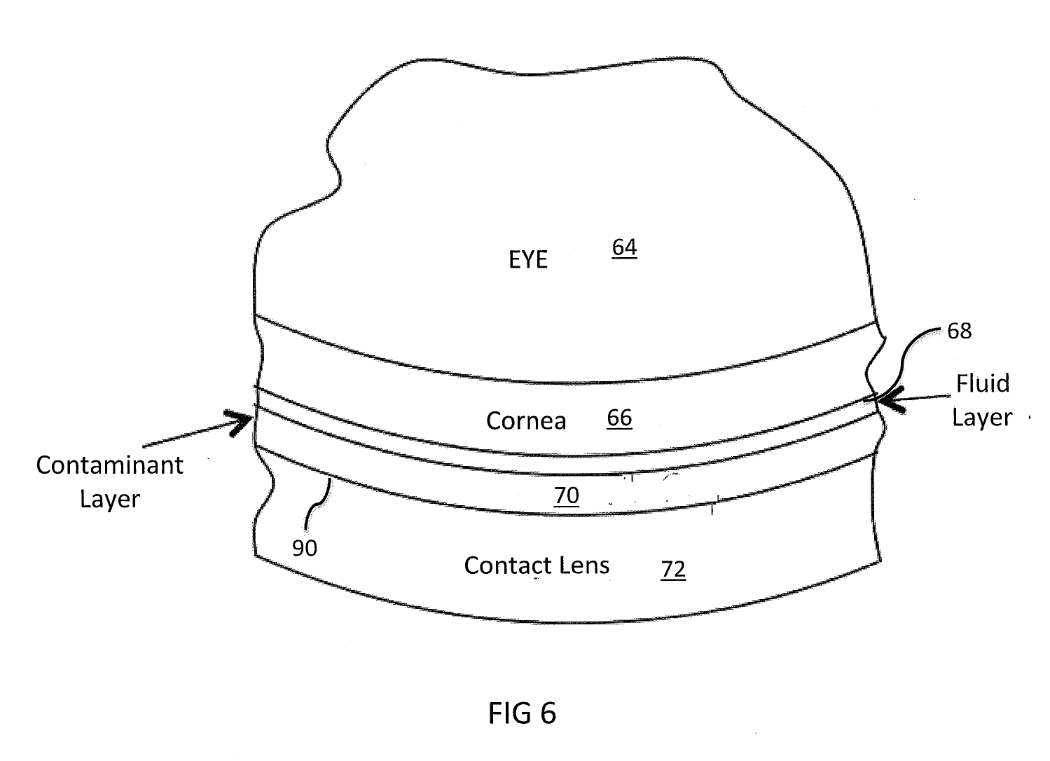

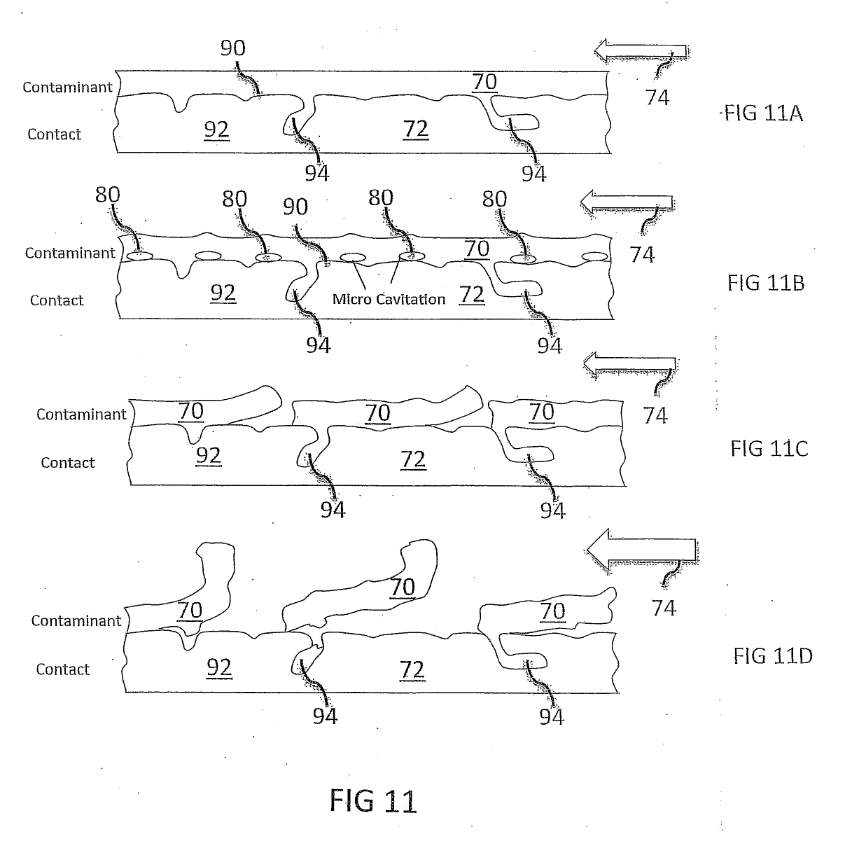

Referring to FIG. 6, while referring generally to FIGS. 6-11, where FIG. 11 includes FIG. 11A-11D, a schematic illustration of an eye 64 with the cornea 66 at the front thereof may bind by a fluid layer 68 a contact lens 72. The surface tension in the fluid layer 68 will tend to hold the lens 72 against the eye 64. Initially a new contact lens 72 will adhere to the eye 64 of a user over the cornea 66 by virtue of the surface tension of the fluid layer 68. Over time, a contaminant layer 70 may develop in the surface or on the surface of the lens 72, resulting in the rather tough and mechanically relatively strong contaminant layer 70.

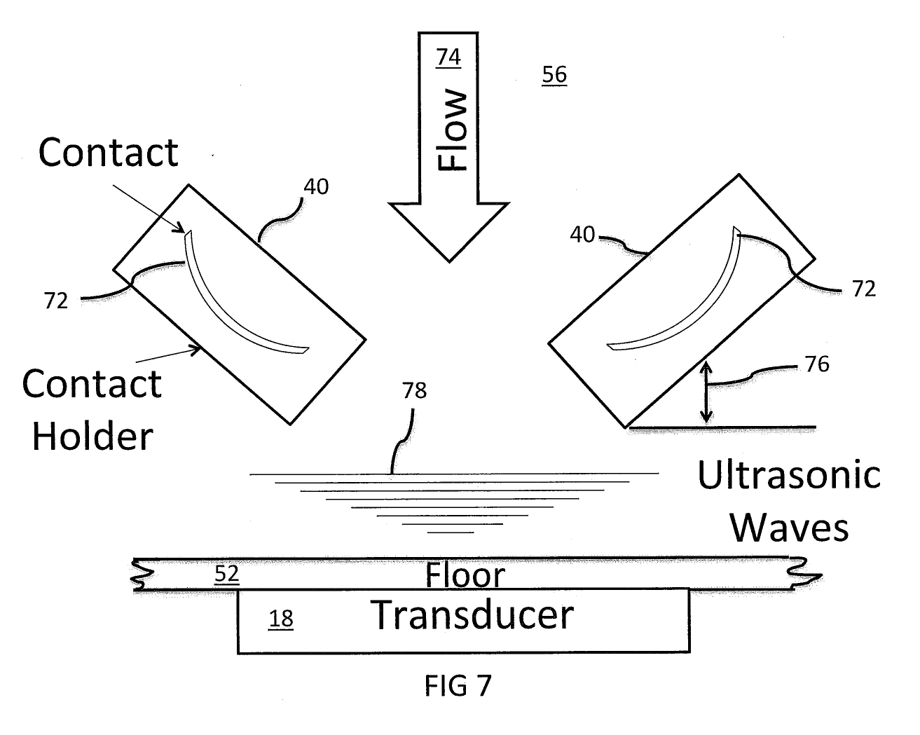

Referring to FIG. 7, while continuing to refer generally to FIGS. 1-11, a lens 72 may be placed in a basket 40 in the apparatus 10 or system 10 in accordance with the invention. In the illustrated embodiment, the flow 74 is schematically illustrated driven by the impeller 46 on the shaft 48 powered by the motor 50. The flow 74 is directed to flow across a surface of each of the lenses 72. Meanwhile, the transducer 18 transmits ultrasonic vibrations into and through the floor 52 of the well 16.

Typically, the baskets 40 are set at an angle 76 with respect to the floor 52. It has been found that functioning of the convective flow 74 operates better when the baskets 40 are not aligned vertically nor horizontally with respect to the floor 52. Thus, for example, an angle from about 30 to about 60 degrees has been found suitable. Typically, the angle 76 may be at about 45 degrees with respect to the floor 52.

In the illustrated embodiment, the ultrasonic waves 78 in the fluid surrounding the baskets 40 and contact lenses 72 propagate throughout the cavity 56 of the well 16. The liquid in the cavity 56 propagates the ultrasonic waves 78, tending to create microcavitation on the surfaces of the lenses 72.

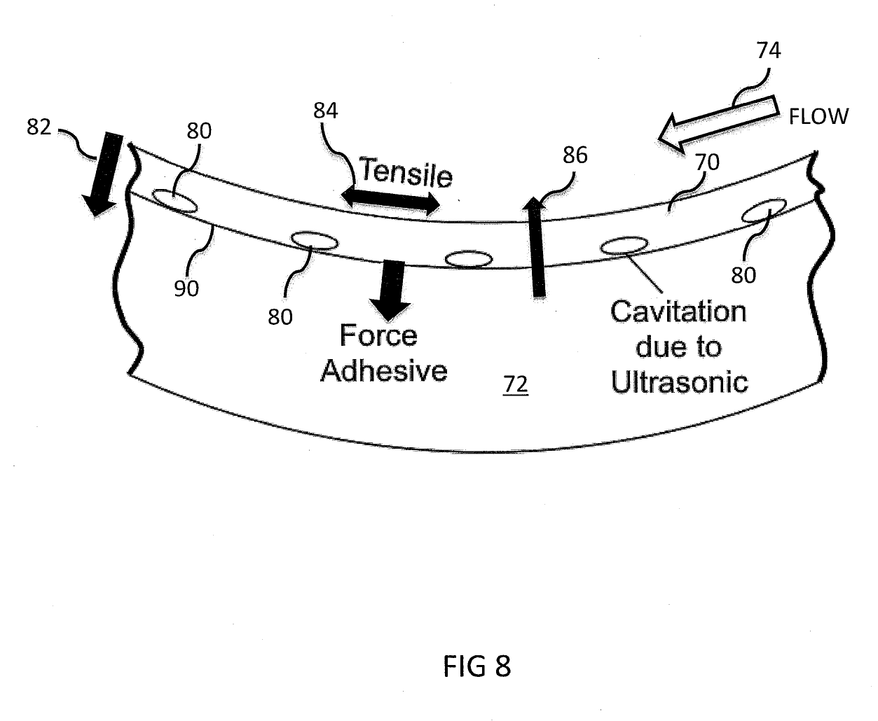

Referring to FIG. 8, while continuing to refer generally to FIGS. 1-11, a schematic illustration of a lens 72 and the attached contaminant layer 70 illustrates cavitation locations 80 or microcavitation bubbles 80 formed at the surface of the contact lens 72 where the contaminant layer 70 attaches. The adhesive force 82 of the contaminant layer with respect to the lens 72 is substantial. It has been found that microorganisms create a structure and the contamination layer 70 has structural integrity sufficient to support tensile forces 84 throughout the contaminant layer 70.

Also, the adhesive force 82 of the contaminant layer 70 binding to the lens 72 is appreciable and not overcome easily by the abrasion of washing. In contrast, the microcavitation bubbles 80 provide a force 86, tending to push the contaminant layer 70 away from the surface 90 to which the layer 70 attaches. However, it has been found that ultrasonic waves alone are not particularly effective at permanently separating the contaminant layers 70 from the surface 90.

The cavitation bubbles 80 indeed originate and expand, thus providing a force 86 tending to separate the layer 70 from the surface 90. However, microcavitation, like all cavitation, is a cyclic process in which bubbles 80 may form but may likewise collapse back on themselves.

Thus, it has been found that the flow 74, or the convective flow 74 driven by the impeller 46 along the surface 90 of the contact lens 72, provides an additional benefit. To the extent that microcavitation bubbles 80 may rupture the contaminant layer 70, the convective flow 74 may urge the discontinuity in the contaminant layer 70 to separate from the surface 90 of the lens 72.

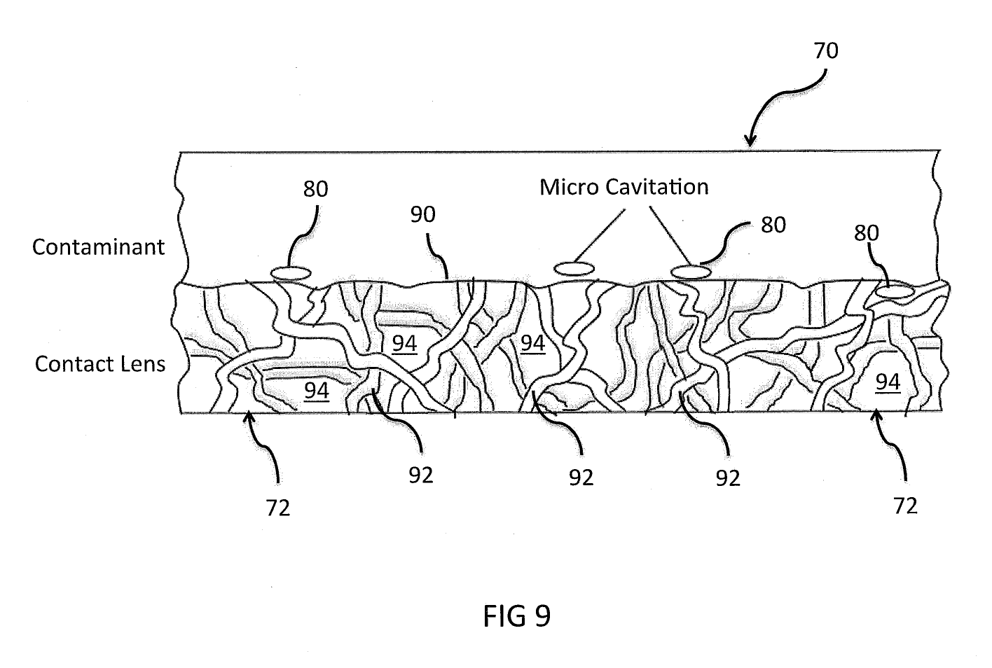

Referring to FIGS. 9-11, where FIG. 11 constitutes FIGS. 11A-11D, the micro structure in a contact lens 72 and its overlying contaminant layer 70 connected to its surface 90 is presented schematically. As a practical matter, polymer chains are not entirely solid.

At a molecular level, polymer strands 92 are formed leaving interstitial spaces 94 therebetween. The microorganisms that form the contaminant layer 70 exist within the interstitial spaces 94. Thus, it is the mechanical structure of colonies of microbes that bind the contaminant layer 70 to the surface 90 by anchoring within the mechanical declivities, the like, interstitial spaces 94, and thus anchoring to the polymer strands 92, themselves.

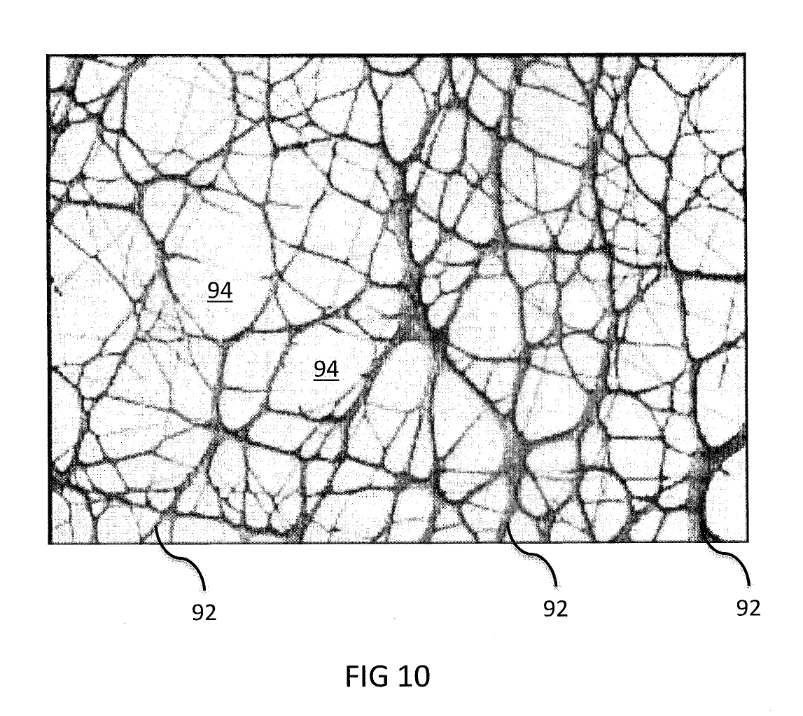

FIG. 10 represents an image at a layer, as seen in a scanning electron micrograph. Of course, many layers of polymeric web 92 or polymer strands 92 will exist within an actual lens 72. Nevertheless, one can see the stranded nature of the polymer.

Referring to FIG. 11A, while continuing to refer generally to FIGS. 1-11, a convective flow 74 flowing over a contamination layer 90 may initially have little effect. Likewise, mechanical abrasion, cleansers, rubbing, and the like by users are not particularly effective against the contamination layer 70. One surface passing against another in the presence of a liquid tends to be lubricated, minimizing friction and shear forces, and thus providing little if any tensile force on a contaminant layer in any direction.

In FIG. 11A, the convective flow 74 passes over the contamination layer or contaminant layer 70, but the contaminant layer 70 is adhered to the contact lens by the growth of the contaminant layer 70 into the interstitial spaces 94 in the polymer 92. Thus, the convective flow 74 by itself may not be particularly effective at removing the contaminant layer 70. Similarly, mechanical abrasion, scrubbing, or rubbing, as mentioned, is not particularly effective.

Moreover, the liquid between a contaminant layer 70 and any scrubbing mechanism, including brushes, fingers, or the like, tends to provide lubricity between the contaminant layer 70 and the implement of abrasion. If an abrasive material is sufficiently hard or effective to penetrate into the layer 70, it is likewise hard enough to damage the surface 90 of the lens 72, rendering it clouded and unsuitable for use. Moreover, any of the soft contact lenses or gas-permeable lenses tend to be light, mechanically soft, and may be ripped and thereby destroyed quite easily.

Referring to FIG. 11B, as microcavitation bubbles 80 begin to form on the surface 90 of a contact lens 72, the contaminant layer 70 may separate locally. Eventually, as illustrated in FIG. 11C, the contaminant layer 70 will rupture. Typically, due to the mechanical structure of the contaminant layer 70 and its embedding within the cavities 94 within the polymer 72, the breaks in the contaminant layer 70 will typically return to their original place. If left they may completely re-attach. However, by vigorous action of the convective flow 74 as illustrated in FIG. 11C, the convective flow 74 eventually begins to operate on the contaminant layer 70, flowing between the contaminant layer 70 and the surface 90, and also acting against the larger surface area exposed by the layer 70.

Referring to FIG. 11D, the pieces of the contaminant layer 70 have been found to be mechanically separated by the convective flow 74. This action takes advantage of the microcavitation bubbles 80 to initially provide a degree of mechanical separation between the layer 70 and the contact lens 72. Then, relying on the fluid drag of a vigorous convective flow 74, a mechanically produced drag force is strong enough to separate and tear loose the large area of the contaminant layer 70 presented to the convective flow 74.

Thus, the combination is most effective. Ultrasonic waves 78 cause microcavitation bubbles 80, lifting the layer 70 sufficiently to break it and break it free locally. This initiation is augmented dramatically by the strong convective flow 70 lifting, pushing and proceeding to tear the layer 70 loose from the lens 72. Thus, the experimental evidence shows that the film 70 or layer 70 of contaminants is torn off the lens 72 by the convective flow 74 much more effectively than, and in a way that cannot be done by, ultrasonic waves 78 acting alone.

In sum, contact lenses are commonly cleaned using prior art systems and methods on a regular schedule to prevent protein deposits and other microbial growth and contaminations. However, most previously known cleaning techniques simply delay progress of the layer 70, introduce harsh chemicals harmful to the eye, or both. Such may result in longer wear, but no real rollback of the effects of contamination. Eye infections still occur with use of prior art systems, some with devastating effect, including loss of vision, or loss of an eye due to infection.

An apparatus and method in accordance with the invention were operated for cleaning and removing contaminants 80 from most substrates 72, particularly including contact lenses 72. Water in a saline solution was the fluid used, in the present design, and chemical additives were included for wetting, reducing interfacial energy, adjusting pH, and disinfecting, thus prolonging the life of contact lenses. The system services all types of lenses such as permanent, disposable, hard, soft, and gas-permeable lenses.

Contaminants deposited on the surfaces developed as a growth of microbes, which tend to secure to the lens by propagating their mechanical structure into the apertures 94 in the matrix 92 of the substrate 72. It was difficult to remove these contaminants without damaging the surface through aggressive cleaning, rubbing or harsh chemicals. Even ultrasonic cleaning did not provide clean lenses. Current technologies in ultrasonic or other subsonic agitations have proven ineffective, both in frequencies used and the lack of any hydrodynamic shear using cross-flow turbulence as induced in the system 10 in accordance with the invention.

The present invention was operated as a contact lens cleaning device at selected ultrasonic frequencies. Induced shear flows due to convective flows in the turbulent domain of Reynolds numbers, were applied to clean the contained contact lenses 72. In certain experiments, a cleaning solution was used to lower interfacial energy of the bonds between contaminants 70 and the contact lenses 72.

It has been found that three main components are involved in the reducing of contaminants and their removal. Initially, ultrasonic waves cause high and low pressure points throughout the substrate 72, contamination layer 70, and at the substrate-contaminant interface 90. The substrate layer 72 and contaminant layer 70 have different physical properties, such as elastic modulus. Thus, the interface therebetween tends to provide cavitation bubbles 80.

These low and high pressure points or areas occur along the interface, and may occur internally in the lens 72 and contaminant layer 70. Changes in pressure and temperature due to ultrasonic waves leads to microcavitation bubbles forming and collapsing along the interface surface 90 of the contact lens 72.

Vibrations induced from ultrasonic waves led to fractures or localized rupture of the contamination layer 70. It is believed that pressure differentials between the substrate 72 and contaminant layers 70 apply low-cycle, high-load fatigue to the interfacial bonds between the substrate 72 and contaminants 70. It is more than just the adhesion of the contaminant layer 70 to the lens 72, but the mechanical strength of the actual biological structures created by microbes that embed in the interstices 94 in the polymer matrix 92 of the lens 72. The structures maintain uncanny integrity, and separate as macroscopic films, visible in the cleaning fluid, rather than as particulate contaminants.

Micro-cavitation 80 along the surface 90 or interface assists in separation and segregation, while a chemical agent facilitates the segregation by lowering the interfacial energy. However, a major influence in taking advantage of localized separation was shear from fluid drag acting against the initially separated or ruptured portions of the contaminant layer 70. This was obtained by cross-flows operating in the turbulent flow regime.

The result was reduced thickness of the hydrodynamic shear boundary layer. Thus any irregularity caused by microcavitation, breakage, and so forth was immediately amplified by applying fluid drag, causing shear forces to the parts of the contaminant layer extending out into the turbulent boundary layer and the bulk flow of the convective stream. The power of fluid drag then engaged and detached the compromised regions, tearing even larger fragments of the layer 70 from the substrate 72 by using the tensile strength of the film 70.

The construction of the housing 12 and lid 30 was of a rigid plastic material. It is contemplated that any suitable plastic, stainless steel, other metal, ceramic or similar material would likewise serve this purpose. The shape of the design of the lid 30 can either match the profile of the overall housing 12, or simply fit the cover 28 around the well 16, either inset into the cover 28 or overlapping, as here, to form the outer body contour of the housing 12.

The illustrated embodiment used a housing 12 generally circular with a circular well 16, although a circular, oval, square or other cross section should serve. Nevertheless, a circular cross section provides the most efficient use of operating fluid in the cavity 56 of the well 16.

In further detail, the well 16 was designed to hold the cleaning solution. It was cylindrical, and spaced away from the outer wall of the housing. It was plastic in the original embodiment but may be made of stainless steel, plated steel, rigid plastic or any other similarly rigid material. Certain soft polymers may be used for soft housing 12.

The cage 38 and the contact holding basket 40 were designed to submerge into a cleaning solution used to disinfect the lens 72 and lower interfacial energy and surface tension. The cleaning solution held in the well 16 was filled to a level above the impeller 46. The height of the liquid level affects how vigorous the flow 74 moves past the lens 72, and may be selected to provide a turbulent Reynolds number.

The baskets 40 were constructed of a rigid plastic material, but may be formed of plastic, metal, ceramic or other similar materials, with one to hold a left and one to hold a right contact separately in place in the same solution bath. The baskets were attached to a cage 38 covering the impeller 46 and drive shaft 48. The cage 38 was secured to the lid 30 and extended down into the fluid in use.

The impeller 46 may be of any suitable material, including silicone and soft polymers. In experiments, a rigid material was used. Thus steel, aluminum, plastic or any other suitable rigid material may serve. The impeller 46 was attached to an electric motor 50 built into the lid 30 to make the assembly 14.

Ultrasonic agitation was achieved using an piezoelectric, ultrasonic transducer 18 attached to the well 16. A piezoelectric transducer 18 has a suitable frequency response, but a speaker or any other electrically powered device 18 capable of producing suitable ultrasonic frequencies may be used. The attachment of the transducer 18 to the well 16 was by a bonding agent, and may be by any suitable bonding agent, including adhesive, glue or welding technology.

The cavity 26 was to be filled with a liquid for cooling or heating the well 16, but liquid was found unnecessary. Thus, the cavity provided principally a degree of sound isolation

Within the housing 12, a cavity 20 separated from the fluid cavity 16 or well 16 was designed to hold power conditioning, electronics, circuit boards, or the like to control and power the DC motor and the transducer 18. AC power may be used directly from a power cord 60 to supply line voltage from an electrical outlet.

The advantages of the present invention include its ability to clean contact lenses 72 of accumulated proteins and other contaminant deposits 70 built, grown, or both onto the surface 90 of a contact lens 72. An important result of a method and apparatus in accordance with the present invention is a fast, versatile, effective method to clean and thereby increase the safe and effective operational lifetime of contact lenses.

The cleaner utilized an ultrasonic frequency between 30 kHz to 120 kHz from a piezoelectric transducer 18 directly mounted to the floor 52 of the well 16. Ultrasonic frequencies effective to generate microcavitation were augmented by turbulent convection flows to remove from the lens surface 90 the film 70 of contaminants 70.

The convective shear flow, and even its vigor in the turbulent flow regime appear to be essential for greatest effectiveness and speed of cleaning. The mechanisms and effectiveness were not found in other ultrasonic contact lens cleaner devices.

The turbulence may be produced using any method of propulsion from an impeller attached to a motor, a pump, ducted jet, or other motive driver. By using both suitable ultrasonic energy frequencies and vigorous shear flows, especially if including turbulence, the contaminants 70 were removed from the contact lens 72, without damage to the soft plastic matrix of the contact lens 72.

Eliminating the contaminants 70 from the lens 72, the life of the contact lens 72 can be prolonged, by mechanically undoing contaminant growth into the polymer matrix 92 within the lens 72. Conventional rubbing or cleaning methods introducing large, localized forces that literally wear and tear the polymers strands 92 apart leaving the contact 72 unusable were proven unnecessary. The present invention provides more energy where actually needed, without the overwhelming mechanical loading where it can damage the lens 72.

While the foregoing written description of the invention enables one of similar technological knowledge to make and use what is considered presently to be the best mode thereof, those of similar skills will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

What is claimed is:

1. A method for cleaning contact lenses, the method comprising:
providing a basket for containment;
providing a lens having a contaminant layer developing thereon;
placing the lens in the basket;
placing the basket in a container having a wall and containing a liquid;
creating ultrasonic waves in the liquid by operating an ultrasonic transducer moving the wall;
passing a convective flow across a surface of the lens;
carrying contaminants from the lens by the convective flow.

2. The method of claim 1, wherein the convective flow is in a turbulent regime.

3. The method of claim 2, wherein the ultrasonic waves are selected to be effective to create micro-cavitation lifting the contaminant layer away from the lens.

4. The method of claim 3, wherein the convective flow is effective to create hydrodynamic drag against a portion of the contaminant layer separated by the ultrasonic waves from the lens.

5. The method of claim 4, wherein the ultrasonic waves are effective to create micro-cavitation urging the contaminant layer away from the lens.

6. The method of claim 5, wherein the hydrodynamic drag is selected to engage the tensile strength of the contaminant layer to a degree effective to separate the contaminant layer from the surface of the lens.

7. The method of claim 6, wherein the contaminant layer is bonded to the lens by mechanical extension of the contaminant layer into the matrix of a polymer forming the lens.

8. The method of claim 7, wherein the ultrasonic waves are effective to create bubbles forcing the contaminant layer from the lens.

9. The method of claim 8, further comprising separating a first portion of the contaminant layer from a second portion by the bubbles creating tensile stresses in the contaminant layer.

10. The method of claim 9, further comprising separating the first portion of the contaminant layer from the lens by the hydrodynamic drag creating tensile stresses in the contaminant layer.

11. An apparatus for cleaning contact lenses:
a well containing a liquid;
a basket submerged in the liquid and holding an article having a surface;
a contaminant layer disposed as a contiguous film on the surface and bonded thereto;
a transducer creating waves in the liquid, the waves impinging on the surface;
a convector creating convection current passing along the surface;
the transducer breaking loose the bonding of the contaminant layer by cavitating the liquid proximate the surface; and
the convector, tearing the contaminant layer from the surface by applying fluid drag to a portion of the contaminant layer extending into the convection current.

12. The apparatus of claim 11, wherein the convection current is flowing in a turbulent flow regime.

13. The apparatus of claim 12, wherein the frequency of the waves is ultrasonic.

14. The apparatus of claim 11, wherein the frequency of the waves is from about 30 to about 120 kilohertz.

15. The apparatus of claim 11, wherein the convector further comprises a motor and impeller, the motor connected to drive the impeller.

16. The apparatus of claim 15, wherein the frequency of the waves is ultrasonic.

17. The apparatus of claim 16, wherein the frequency is from about 30 to about 120 kilohertz.

18. The apparatus of claim 17, wherein the liquid comprises water and a cleaning agent selected from a surfactant, a bactericide, and a salt.

19. The apparatus of claim 11, wherein:
the lens is formed of a polymer having strands defining interstices therebetween; and
the contaminant layer extends into the interstices.

20. A method of cleaning contact lenses comprising:
providing a housing;
providing a well within the housing;
providing a convection drive assembly within the well;
providing baskets to hold contact lenses;
providing a convective impeller effective to motivate a vigorous convective flow along a surface of a contact lens captured in a basket;
providing an ultrasonic transducer generating ultrasonic waves in a liquid surrounding the baskets;
operating simultaneously the ultrasonic generator and the convective drive assembly to impose microcavitation between a contaminant layer adhered to a contact lens and the surface of contact lens; and
creating a convective shear flow imposing hydrodynamic drag on exposed projections of the contaminant layer to a degree effective to tear the contaminant layer from the surface of the contact lens upon exposure due to microcavitation between the layer and the contact lens.
